(19) United States
(12) Patent Application Publication
(10) Pub. No.: US 2019/0127456 A1
(43) Pub. Date: May 2, 2019

(54) METHODS OF TREATING IDIOPATHIC PULMONARY FIBROSIS

(71) Applicant: FibroGen, Inc., San Francisco, CA (US)

(72) Inventors: Seth Porter, San Carlos, CA (US); Kenneth E. Lipson, San Mateo, CA (US); Eduard Gorina de Travy, Belmont, CA (US); Kin-Hung Peony Yu, Hillsborough, CA (US)

(73) Assignee: FibroGen, Inc., San Francisco, CA (US)

(21) Appl. No.: 16/132,150

(22) Filed: Sep. 14, 2018

Related U.S. Application Data
(60) Provisional application No. 62/579,729, filed on Oct. 31, 2017.
(60) Provisional application No. 62/614,882, filed on Jan. 8, 2018.

(51) Int. Cl. C07K 16/22; A61P 11/00

(52) U.S. Cl. CPC C07K 16/22; A61K 2039/505; C07K 2317/24; A61P 11/00

(57) ABSTRACT

The present invention relates to methods and medicaments useful for treating idiopathic pulmonary fibrosis (IPF) by administering an anti-CTGF antibody. In particular, the treatment methods provided avoid toxicities associated with approved therapies and also avoid the attenuation of the efficacy of an anti-CTGF antibody caused by these approved therapies.

### CROSS REFERENCE TO RELATED APPLICATIONS

This application claims the benefit under 35 U.S.C. § 119(e) of U.S. Provisional Application 62/579729 filed 31 Oct. 2017 and United States Provisional Application 62/614,882 filed 8 Jan. 2018 and are hereby incorporated by reference in their entirety.

### FIELD OF THE INVENTION

The present invention relates to methods and medicaments useful for treating idiopathic pulmonary fibrosis.

### BACKGROUND OF THE INVENTION

Idiopathic pulmonary fibrosis (IPF) is a chronic and progressive lung disease that results in respiratory failure and death. Median survival is about 2 to 4 years from diagnosis. The etiology of IPF remains unknown, but the disease is characterized by fibrotic interstitial infiltrates that are consistent with the histopathologic pattern of usual interstitial pneumonia. (Gross T J et al. N Engl J Med (2001) 345:(7):517-525.) As interstitial fibrosis advances with accompanying distortion of lung architecture, the lung becomes less compliant, increasing the effort associated with breathing, leading to dyspnea. Typically, lung function declines slowly over time, but some patients experience rapid declines that can lead to hospitalization or death, particularly in later stages of the disease. (Martinez F J et al. Ann Intern Med (2005) 142:963-967.)

In the United States, as many as 89,000 people are afflicted with IPF, with about 34,000 newly diagnosed annually. (Raghu G et al., Am J Respir Crit Care Med (2006) 174: (7):810-816.) Prevalence of IPF ranges from 14.0 to 42.7 cases per 100,000 persons and the annual incidence ranges from 6.8 to 16.3 cases per 100,000 persons, depending on the strictness of the diagnostic criteria employed. (Raghu G et al., supra.) The prevalence of IPF increases with age, with most IPF patients 60 years of age or older at the time of diagnosis. The disease is more common in men than in women (Fernandez Perez E R et al. Chest (2010) 137:(1):129-137) with most patients current or former smokers. A familial form of IPF may account for as many as 20% of IPF cases. (Loyd J E, Eur Respir Rev (2008) 17:(109):163-167.)

While the pathogenesis of IPF is not clearly defined, the disease is believed to be caused by repetitive epithelial injury. (Selman M et al. Ann Intern Med (2001) 134:136-151; Selman M. Proc Am Thorac Soc (2006) (4):364-372.) According to this hypothesis, alveolar cell injury and activation initiate a dysregulated, exaggerated fibrotic healing process characterized by myofibroblast proliferation and progressive deposition of extracellular matrix (ECM) in genetically susceptible individuals. (Selman M et al. (2001) supra; Selman M. (2006) supra.)

Recently two new drugs, pirfenidone and nintedanib, have been approved in the United States and other jurisdictions for the treatment of IPF. Pirfenidone, 5-methyl-1-phenyl-2-(1H)-pyridone, is an anti-fibrotic and anti-inflammatory (U.S. Pat. Nos. 7,566,729; 8,609,701; 7,635,707; 7,988,994; 8,383,150; and 5,310,562). Nintedanib is a substituted indolinone inhibitor of receptor tyrosine kinases (U.S. Pat. Nos. 6,762,180; 7,119,093; 7,989,474). The use of these drugs is unfortunately associated with serious side effects, including hepatotoxicities, photosensitivity, skin rash and gastrointestinal disorders, that can cause patients prescribed these medication to discontinue their use. Further, these approved drugs, at best, can only blunt the progression of IPF.

The progressive and fatal nature of IPF, coupled with the often unacceptable side effects associated with the use of pirfenidone and nintedanib underscore the need for improved methods and agents to treat this devastating disease. The present invention meets this unmet medical need by providing novel methods that can reduce, stabilize, or reversing the progression and severity of IPF while avoiding the toxicities associated with the use of pirfenidone and nintedanib. Further, the methods provided herein, avoid the attenuation of the efficacy of an anti-connective tissue growth factor (CTGF) antibody by pirdenidone and nintedanib.

### SUMMARY OF THE INVENTION

In one aspect of the invention, a method is provided for treating IPF in a subject in need thereof, wherein the method comprises administering to the subject an effective amount of an anti-CTGF antibody, without the concomitant use of pirfenidone and/or nintedanib, thereby treating IPF. The avoidance of concomitant treatment of an anti-CTGF antibody with pirfenidone and/or nintedanib prevents the unexpected attenuation of the therapeutic benefits of the anti-CTFG antibody.

In one embodiment, the invention provides a method for treating idiopathic pulmonary fibrosis (IPF) in a subject in need thereof, previously treated with pirfenidone and/or nintedanib. Using this method, pirfenidone and/or nintedanib administration is discontinued at least 2 days prior to the administration of an anti-CTGF antibody to the subject, thereby treating the subject's IPF.

In some embodiments, the method for treating IPF with the avoidance of the concomitant treatment with pirfenidone and/or nintedanib reduces the pathologic rate of decline of a pulmonary function parameter. In other embodiments, the method of treating IPF with the avoidance of concomitant treatment with pirfenidone and/or nintedanib stabilizes or improves (reverses) the pathologic decline of a pulmonary function parameter. Typically, the pathologic rate of decline is compared to a subject's baseline measurement or historic controls. In further embodiments, the pulmonary function parameter is selected from the group consisting of vital capacity (VC), residual volume (RV), forced expiratory volume (FEV), forced vital capacity (FVC), forced vital capacity percent predicted (FVCPP), forced expiratory flow (FEF), peak expiratory flow rate (PEFR), inspiratory reserve volume (IRV), functional residual capacity (FRC), inspiratory capacity (IC), total lung capacity (TLC), expiratory reserve volume (ERV), tidal volume (TV), and maximum voluntary ventilation (MVV).

In other embodiments, the method for treating IPF with the avoidance of the concomitant treatment with pirfenidone and/or nintedanib comprises stabilizing or producing at least a 2% reduction, compared to a subject's baseline measurement or historic controls, in one or more pulmonary radiographic parameters selected from the group consisting of ground glass opacities, parenchymal fibrosis, and honeycomb formation.

In some embodiments, the treatment method comprises the use of an anti-CTGF antibody that has the same amino acid sequence as the antibody produced by the cell line identified by ATCC Accession No. PTA-6006. In other embodiments, the anti-CTGF antibody binds to CTGF competitively with an antibody produced by the cell line identified by ATCC Accession No. PTA-6006. I n certain embodiments, the anti-CTGF antibody is pamrevlumab.

In some embodiments, the method for treating IPF comprises administering at least about 30 mg/kg of an anti-CTGF antibody without the concomitant administration of pirfenidone and/or nintedanib. In other embodiments, the method for treating IPF further comprises administering an additional therapeutic agent selected from the group consisting of corticosteroids, antibiotics, immunosuppressive drugs, supplemental oxygen, and mechanical ventilation.

In a further aspect, the invention provides a method of treating IPF in a subject in need thereof, with an improved gastrointestinal safety profile, comprising administering an effective dose of an anti-CTGF antibody, wherein the improved gastrointestinal safety profile of the method is in comparison to current approved IPF therapies (nintedanib and/or pirfenidone).

In an additional aspect, the invention provides a method for improving the quality of life of a subject with IPF, stabilizing the rate of decline in the quality of life, or reducing the rate of decline in the quality of life. The method comprises administering an effective dose of an anti-connective tissue growth factor (CTGF) antibody to the subject, thereby improving the quality of life, stabilizing the rate of decline in the quality of life or reducing the rate of decline in the quality of life of the subject. In some embodiments, the subject's quality of life is measured by a self-administered questionnaire. In further embodiments, the self-administered questionnaire is the St. Georges Respiratory Questionnaire (SGRQ) or the University of California, San Diego Shortness of Breath Questionnaire (UCSD-SOBQ).

These and other embodiments of the present invention will readily occur to those of skill in the art in light of the disclosure herein, and all such embodiments are specifically contemplated.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

### BRIEF DESCRIPTION OF THE DRAWINGS

FIG. 1 illustrates the lung density, measured in Hounsfield Units (HU), of mice irradiated with 14.5 Gy photons but otherwise untreated after 15 weeks and after 24 weeks. Dashed line indicates the lung density of non-irradiated mice at the same time periods.

FIG. 2 illustrates the lung density, measured in Hounsfield Units (HU), of mice irradiated with 14.5 Gy photons and treated as indicated for 8 weeks starting at 16 weeks after irradiation. Dashed line indicates the lung density of irradiated mice at 15 weeks. RT is radiation; hIgG is human IgG immunoglobulin; PF is pirfenidone; ND is nintedanib; FG is pamrevlumab.

FIG. 3 illustrates the lung volume (cm³) of mice irradiated with 14.5 Gy photons but otherwise untreated after 15 weeks and after 24 weeks. Dashed line indicates the lung volume of non-irradiated mice at the same time periods.

FIG. 4 illustrates the lung volume (cm³) of mice irradiated with 14.5 Gy photons and treated as indicated for 8 weeks starting at 16 weeks after irradiation. Dashed line indicates the lung volume of irradiated mice at 15 weeks. RT is radiation; hIgG is human IgG immunoglobulin; PF is pirfenidone; ND is nintedanib; FG is pamrevlumab.

FIGS. 5A-5D illustrate changes from baseline in three domains (FIG. 5A, symptoms; FIG. 5B, activity; and FIG. 5C, impact) and in total score (FIG. 5D) for the St. George's Respiratory Questionnaire from subjects in a double-blind, placebo-controlled Phase two study of an anti-CTGF antibody (pamrevlumab) for the treatment of IPF. The score for each domain ranges from 0 to 100, with a higher score indicating a worse health-related quality of life parameter.

FIG. 6 illustrates changes from baseline in the University of California, San Diego—Shortness of Breath Questionnaire (UCSD-SOBQ) from subjects in a double-blind, placebo-controlled Phase two study of an anti-CTGF antibody (pamrevlumab) for the treatment of IPF. The UCSD-SOBQ is a self-reported measure of dyspnea as assessed by 24 sections that evaluate dyspnea associated with activities of daily living (ADLs). Each question has a 6-point scale (0=“not at all” to 5=“maximal or unable to do because of breathlessness.” The total score ranges from 0 to 120, with higher scores indicating greater dyspnea.

FIG. 7 is a scatter plot and Spearman correlation between the UCSD-SOBQ score and the SGRQ activity domain score for subjects in a double-blind, placebo-controlled Phase two study of an anti-CTGF antibody (pamrevlumab) for the treatment of IPF.

FIG. 8 is a scatter plot and Spearman correlation between the UCSD-SOBQ score and the SGRQ total score for subjects in a double-blind, placebo-controlled Phase two study of an anti-CTGF antibody (pamrevlumab) for the treatment of IPF.

FIG. 9 is a scatter plot and Spearman correlation between the UCSD-SOBQ score and FVCPP for subjects in a double-blind, placebo-controlled Phase two study of an anti-CTGF antibody (pamrevlumab) for the treatment of IPF.

### DESCRIPTION OF THE INVENTION

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications cited herein are incorporated herein by reference in their entirety for the purpose of describing and disclosing the methodologies, reagents, and tools reported in the publications that might be used in connection with the present invention. Nothing herein is to be construed as an admission that the present invention is not entitled to antedate such disclosure by virtue of prior invention.

The practice of the present invention will employ, unless otherwise indicated, conventional methods of chemistry, biochemistry, molecular biology, cell biology, genetics, immunology and pharmacology, within the skill of the art. Such techniques are explained fully in the literature. See, e.g., Gennaro, A. R., ed. (1990) Remington's Pharmaceutical Sciences, 18th ed., Mack Publishing Co.; Colowick, S. et al., eds., Methods In Enzymology, Academic Press, Inc.; Handbook of Experimental Immunology, Vols. I-IV (D. M. Weir and C. C. Blackwell, eds., 1986, Blackwell Scientific Publications); Maniatis, T. et al., eds. (1989) Molecular Cloning: A Laboratory Manual, 2nd edition, Vols. I-III, Cold Spring Harbor Laboratory Press; Ausubel, F. M. et al., eds. (1999) Short Protocols in Molecular Biology, 4th edition, John Wiley & Sons; Ream et al., eds. (1998) Molecular Biology Techniques: An Intensive Laboratory Course, Academic Press); PCR (Introduction to Biotechniques Series), 2nd ed. (Newton & Graham eds., 1997, Springer Verlag).

### Definitions

As used herein, the term “about” refers to ±10% of the numerical value of the number with which it is being used. Therefore, about 50% means in the range of 45%-55%.

As used herein, the singular form “a,” “an,” and “the” include plural references unless the context clearly dictates otherwise. For example, a reference to “an anti-CTGF antibody” includes a plurality of such antibodies and to equivalents thereof known to those skilled in the art; and so forth.

As used herein, the term “subject,” “host,” “individual,” and “patient” are used interchangeably to refer to a mammal. In a preferred embodiment, the mammal is a primate, and more preferably a human being.

As used herein, the term “blood” encompasses whole blood, serum or plasma. When a specific antibody concentration in plasma, e.g., a target antibody plasma level, is discussed, it is to be understood to include the antibody concentration in whole blood, serum or plasma.

The terms “idiopathic pulmonary fibrosis” and “IPF” describe a chronic, progressive fibrosing interstitial pneumonia of unknown cause, limited to the lungs and associated with the radiologic and/or histopathologic pattern of usual interstitial pneumonia (UIP).

Subjects with IPF have a UIP pattern on high resolution computerized tomography (HRCT) scan with the following three features: (1) subpleural, basal predominance of fibrosis; (2) reticular abnormality; and (3) presence of honeycombing with or without traction bronchiectasis. Additionally, IPF subjects do not have any of the following features inconsistent with an UIP pattern: (i) upper or mid-lung predominance of fibrosis; (ii) peribronchovascular predominance fibrosis; (iii) extensive ground glass abnormality (extent>reticular abnormality); (iv) profuse micronodules (bilateral, predominately upper lobes); (v) discrete cysts (multiple, bilateral away from areas of honeycombing); (vi) diffuse mosaic attenuation/air trapping (bilateral, in three or more lobes); and (vii) consolidation in bronchopulmonary segment(s) and/or lobe(s). These criteria represent the official statement of the American Thoracic Society (ATS), The European Respiratory Society (ERS), The Japanese Respiratory Society (JRS), And The Latin American Thoracic Association (ALAT). (See Raghu G, et al. Am J Respir Crit Care Med. (2011) 183: (6):788-824.)

Subjects with IPF can also have a possible UIP pattern on HRCT scan with histopathological confirmation of UIP. The subjects have the following two features present on their HRCT scan: (1) subpleural, basal predominance of fibrosis; and (2) reticular abnormality. Additionally, the following features that are inconsistent with a UIP pattern are absent: (i) upper or mid-lung predominance of fibrosis; (ii) peribronchovascular predominance of fibrosis; (iii) extensive ground glass abnormality (extent >reticular abnormality); (iv) profuse micronodules (bilateral, predominately upper lobes); (v) discrete cysts (multiple, bilateral away from areas of honeycombing); (vi) diffuse mosaic attenuation/air trapping (bilateral, in three or more lobes); and (vii) consolidation in bronchopulmonary segment(s) and/or lobe(s). (See Raghu G, et al. supra)

For histopathological confirmation of UIP pattern, the following four criteria are met: (1) evidence of marked fibrosis/architectural distortion, ±honeycombing in a predominantly subpleural/paraseptal distribution; (2) presence of patchy involvement of lung parenchyma by fibrosis; (3) presence of fibroblast foci; and (4) absence of features against a diagnosis of UIP suggesting an alternate diagnosis, e.g., hyaline membranes, organizing pneumonia, granulomas, marked interstitial inflammatory cell infiltrate away from honeycombing, predominant airway centered changes, etc. (See Raghu, supra)

As used herein, the terms “treating”, “treatment,” and “therapy,” in the context of the invention, mean the administration of an anti-CTGF antibody to subjects with IPF or at risk for developing IPF. In some embodiments, the subjects with IPF are responsive to conventional treatment. In other embodiments, the subjects with IPF are non-responsive to conventional treatment or cannot tolerate conventional treatment. In further embodiments, the IPF subjects treated with anti-CTGF antibody are those subjects that are treatment naive and include newly diagnosed IPF subjects.

As used herein, the terms “effective amount” or “therapeutically effective amount” in the context of administering an anti-CTGF antibody to a subject, refer to the amount of an anti-CTGF antibody that is sufficient to produce a beneficial or therapeutic effect including a partial or complete cure of IPF, or the alleviation, amelioration, stabilization, improvement, or reversal of the disease or any associated symptoms of the disease. In some embodiments, an associated symptom of IPF is the pathologic rate of decline in one or more pulmonary function parameters, discussed below. In specific embodiments, an “effective amount” of an anti-CTGF antibody refers to an amount of an anti-CTGF antibody that is sufficient to produce at least one or more of the following effects compared to a baseline measurement, i.e., pretreatment, or a historic control: (i) a reduction in a pathologic rate of decline for one or more pulmonary function parameters; (ii) a stabilization (arrest or stasis) in the pathologic rate of decline in one or more pulmonary function parameters; or (iii) a reversal in pathologic rate of decline in one or more pulmonary function parameters, including the normalization of one or more pulmonary function parameters.

As used herein, the terms “avoidance” and “avoiding” mean “refraining from” or “doing without.” For example, “avoiding concomitant treatment with pirfenidone and/or nintedanib” in the context of the claimed invention, means refraining from treating a subject with one or both of these agents when the subject is to be treated or is currently undergoing treatment with an anti-CTGF antibody. In some embodiments, “avoiding” concomitant therapy with pirfenidone and/or nintedanib means never treating a subject with these agents prior to the treatment with an anti-CTGF antibody. In other embodiments, “avoidance” or “avoiding” concomitant therapy include discontinuing the treatment of pirfenidone and/or nintedanib at least 2 days prior to starting therapy with an anti-CTGF antibody. In various embodiments, pirfenidone and/or nintedanib is discontinued at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, at least 28 days, at least 29 days, at least 30 days, or at least one month, prior to starting therapy with an anti-CTGF antibody.

The experimental results disclosed in the Example section demonstrate that co-treatment with pirfenidone and/or nintedanib attenuates or reduces the efficacy of an anti-CTGF antibody. To avoid attenuation, a “washout” period is required, i.e., a period of time between the cessation of treatment with pirfenidone and/or nintedanib and the initiation of treatment with an anti-CTGF antibody. Typically, regulatory bodies recommend the use of a washout period of at least 5-7 half lives of the discontinued first drug before a subject is switched to second drug. For drugs that follow a one or two compartment open body model, the duration of the washout time of 10x the plasma apparent terminal elimination half-life will provide for 99.9% of the administered dose to be eliminated from the body. In healthy, young Chinese adults, a terminal t_{1/2} of 2 hrs to 2.5 hrs was calculated for pirfenidone (Shi S., et al. J Clin Pharmacol. (2007) 47:1268-1276). Applying a 5×t_{1/2} for pirfenidone yields a minimum washout time of 10-12.5 hrs. A 10×t1/2 washout period is 20-25 hrs. The plasma half-life for nintedanib in IPF patients was 9.5 hrs (OFEV (nintedanib) label). Applying a 5×t1/2 for nintedanib yields a minimum washout time of 47.5 hrs. A 10×t1/2 washout period is 95 hrs. In some embodiments, treatment with an anti-CTGF antibody is not initiated following the cessation of treatment with pirfenidone and/or nintedanib until at least 5 times the terminal t1/2 of the particular drug to have elapsed. In further embodiments, treatment with an anti-CTGF antibody is not initiated until at least 6, at least 7, at least 8, at least 9 or at least 10 times the terminal t_{1/2}of the particular drug to have elapsed. In specific embodiments, for subjects undergoing treatment, or have recently ceased treatment with pirfenidone and/or nintedanib, the administration of an anti-CTGF antibody should not initiated until at least 2 days, at least 3 days, at least 4 days, at least 5 days, at least 6 days, at least 7 days, at least 8 days, at least 9 days or at least 10 days have passed following the subject's last treatment of pirfenidone and/or nintedanib. In particular embodiments, treatment with an anti-CTGF antibody should not be initiated until at least 1 day has elapsed since the cessation of treatment with pirfenidone.

Lung capacity and associated pulmonary function parameters naturally decline due to aging. Numerous normal populations have been studied and the rate of decline of lung capacity and various pulmonary function parameters have been calculated and are readily available in the art. (Crapo et al. (1981) Am. Rev. Respir. Dis. 123:659-664.) For example, a 65 year-old Caucasian male who is 183 cm (6′0″) tall has a predicted FVC of 4.95 liters. At age 66 this same male has a predicted FVC of 4.92 liters. This difference of 0.03 liters represents the expected decline due to aging by 1 year. Similarly, a 62 year-old Caucasian woman who is 167 cm (about 5′6″) has a predicted FVC of 2.67 liters. At age 63, this same female has a predicted FVC of 2.64 liters. This difference of 0.03 liters represents the expected decline due to aging by 1 year.

Numerous pulmonary function parameters known in the art can be used to monitor a patient's response to treatment with an effective amount of an anti-CTGF antibody. These pulmonary function parameters include the following:

Vital capacity (VC) is the total volume of air that can be moved in and out of the lungs. VC is equal to the combined inspiratory reserve volume, tidal volume, and expiratory reserve volume.

Forced vital capacity (FVC) is the vital capacity from a maximally forced expiratory effort.

FVCPP is a subject's measured FVC expressed as the percentage of the predicted FVC for the subject. As used herein, all FVCPP values are absolute values and not relative values.

Residual volume (RV) is the volume of air remaining in the lungs after a maximal exhalation.

Forced expiratory volume (FEV) is the expiratory volume of air from a maximally forced expiratory effort, usually measured over a set period of time, e.g., 1 second, FEV1; 6 seconds, FEV6; etc.

Forced inspiratory flow (FIF) is the inspiratory volume of air from a maximally forced inspiratory effort, usually measured over a set period of time, e.g., 1 second, FIF1; 6 seconds, FIFE; etc.

Peak expiratory flow rate (PEFR) is the highest forced expiratory flow rate.

Inspiratory reserve volume (IRV) is the maximal volume that can be inhaled after a normal inspiration, measured from the end-inspiratory level.

Tidal volume (TV) is the volume of air inhaled or exhaled during one respiratory cycle, typically measured at rest.

Inspiratory capacity (IC) is the sum of the inspiratory reserve volume and the tidal volume.

Functional residual capacity (FRC) is the sum of the expiratory reserve volume and the residual volume. Typically, FRC represents the volume of air in the lungs at the end of a normal expiration.

Total lung capacity (TLC) is the sum of the vital capacity and residual volume that represents the total volume of air that can be contained in the lung.

Expiratory reserve volume (ERV) is the maximal volume of air that can be exhaled after a normal expiration, measured from the end-expiratory position.

Maximum voluntary ventilation (MVV) is the volume of air expired in a specified time period during repetitive maximal effort.

The FEV1/FVC ratio is the ratio between forced expiratory volume in one second and forced vital capacity.

Many of these pulmonary function parameters are readily obtainable through the use of a spirometer as is well-known in the art. Residual volume can be obtained through indirect methods such as radiographic planimetry, body plethysmography, closed circuit dilution (including the helium dilution technique), and nitrogen washout.

In contrast to the natural decline in pulmonary function due to aging, subjects with IPF have an abnormally steep rate of decline i.e., a “pathologic rate of decline,” in lung capacity or in one or more pulmonary function parameters. As used herein, a “pathologic rate of decline” is a rate of decline in lung capacity or in one or more pulmonary function parameters that is at least 1% greater than the decline due to normal aging. In some embodiments, a pathologic rate of decline is at least 1%, 2%, 3%, 4%, 5%, 10%, 15%, 20%, 25%, 30%, 40%, 50%, 60%, 70%, 80%, 90%, 100%, 125%, 150%, 200%, 300%, 400%, 500%, 600%, 700%, 800%, or 1000% greater than the predicted rate of decline for a normal person of similarly matched race or ethnicity, gender, age, height, and weight. Rates of decline can be expressed as the change from a baseline measurement over a chosen time period, e.g., 1 month, 2 months, 4 months, 6 months, 9 months or 12 months.

In some embodiments, a method is provided for reducing, stabilizing, or reversing a pathologic rate of decline in one or more pulmonary function parameters, comprising the administration of an effective amount of an anti-CTGF antibody without the concomitant treatment with pirfenidone and/or nintedanib. Pulmonary function parameters that are typically measured to assess efficacy include parameters selected from the group consisting of VC, RV, FEV, FVC, FVCPP, FEF, PEFR, IRV, FRC, IC, TLC, ERV, TV, and MVV.

In further embodiments, treatment with an effective amount of anti-CTGF antibody without concomitant treatment with pirfenidone and/or nintedanib reduces the pathologic rate of decline of one or more pulmonary function parameters by at least 1%, 2%, 3%, 4%, 5%, 10%, 15%, 20%, 30%, 40%, 50%, 60%, 80%, or 100% compared to a baseline measurement or to historical controls. In particular embodiments, the pulmonary function parameter is FVC or FVCPP. In further embodiments, the reduction, stabilization, or reversal in the pathologic rate of decline is achieved in 3 weeks or less, 6 weeks or less, 9 weeks or less, 12 weeks or less, 18 weeks or less, 24 weeks or less, 36 weeks or less, 48 weeks or less, 12 months or less, 16 months or less, 20 months or less, or 24 months or less from starting treatment.

In some embodiments, a method is provided for increasing a pulmonary functional parameter subject with IPF by administering an effective amount of an anti-CTGF antibody, without the concomitant administration of pirfenidone and/or nintedanib. In particular embodiments, the improvement in pulmonary function parameter is an improvement in FVC. In some embodiments, FVC is increased by at least 0.5%, 1.0%, at least 2.0%, at least 3.0%, at least 4.0%, 5.0%, 6.0%, 7.0%, 8.0%, 9.0%, 10%, 15%, 20%, 30%, 40%, or 50% compared to a baseline measurement or historic controls. In additional embodiments, the method increases FVC in a subject with IPF by at least 0.05 liters, 0.1 liters, 0.15 liters, 0.20 liters, 0.25 liters, or 0.3 liters compared to baseline FVC or historic controls. In further embodiments, increase in the pulmonary function parameter is achieved with in 3 weeks or less, 6 weeks or less, 9 weeks or less, 12 weeks or less, 18 weeks or less, 24 weeks or less, 36 weeks or less, 48 weeks or less, 12 months or less, 16 months or less, 20 months or less, or 24 months or less of initiating treatment.

In other embodiments, the pulmonary function parameter is FVCPP and the method increases FVCPP by at least 0.5%, 1%, 1.5%, 2.0%, 2.5%, 3.0%, 4.0%, 5.0%, 6.0%, 7.0%, 8.0%, 9.0%, 10%, 15%, 20%, 30%, 40%, or 50% compared to baseline FVCPP or historic controls. For example, if a subject with IPF has a baseline FVCPP of 65%, treatment with an anti-CTGF antibody may raise the subject's FVCPP to 66.5% at week 48 post-initiation of therapy. In further embodiments, an increase in FVCPP is achieved in 3 weeks or less, 6 weeks or less, 9 weeks or less, 12 weeks or less, 18 weeks or less, 24 weeks or less, 36 weeks or less, 48 weeks or less, 12 months or less, 16 months or less, 20 months or less, or 24 months or less from initiating treatment.

In some embodiments, treatment with an effective amount of an anti-CTGF antibody without concomitant treatment with pirfenidone and/or nintedanib, is sufficient to produce: (i) an increase in diffusing capacity of the lung for carbon monoxide (DLCO) corrected for hemoglobin compared to baseline, i.e., pretreatment: (ii) an increase in the DLCO percent (DLCO %) predicted compared to baseline; (iii) an increase in arterial oxyhemoglobins saturation (SaO₂) compared to baseline; or (iv) a decrease in alveolar-arterial oxygen tension gradient (A-a) PO₂ compared to a baseline measurement or historic controls. In some embodiments, the increase in DLCO, DLCO % predicted, or SaO₂ is at least 5%, 10%, 15%, 20%, 25%, 30%, 35%, 40%, 45%, 50%, 55%, 60%, 65%, 70%, 75%, 80%, or 90% above a baseline measurement or historic controls. In other embodiments, the decrease in (A-a)PO₂ is at least 5%, 10%, 15%, 20%, 25%, 30%, 35%, 40%, 45%, 50%, 55%, 60%, 65%, 70%, 75%, 80%, or 90% below a baseline measurement or historic controls. DLCO, DLCO % predicted, SaO₂, or (A-a) PO₂ can be measured at rest or after exercise, e.g., the standardized 6-minute walk test. In further embodiments, treatment is sufficient to produce a desired change in DLCO, DLCO % predicted, SaO₂, or (A-a) PO₂ value in 3 weeks or less, 6 weeks or less, 9 weeks or less, 12 weeks or less, 18 weeks or less, 24 weeks or less, 36 weeks or less, 48 weeks or less, 12 months or less, 16 months or less, 20 months or less, or 24 months or less from starting treatment.

In some embodiments, treatment with an effective amount of an anti-CTGF antibody, without the concomitant use of pirfenidone and/or nintedanib, is sufficient to produce a reduction, stabilization, or reversal of at least one or more of the following histopathologic features compared to a baseline measurement or historical controls: (i) degree of pulmonary infiltration of fibroblasts and/or myofibroblasts; (ii) rate of collagen deposition; (iii) degree of type II pneumocyte hyperplasia; (iv) degree of smooth muscle hyperplasia, or (v) formation of fibroblastic foci (buds of young proliferating fibroblasts adjacent to alveoli). Typically, these histopathological features are more commonly seen in subpleural regions of the lower lung zones. In some embodiments, treatment with an effective amount of an anti-CTGF antibody, without the concomitant use of pirfenidone and/or nintedanib, is sufficient to produce a reduction of at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, or 95% in at least one or more histopathologic feature compared to a baseline measurement or historic controls. In further embodiments, the reduction in one or more histopathological feature is achieved in 3 weeks or less, 6 weeks or less, 9 weeks or less, 12 weeks or less, 18 weeks or less, 24 weeks or less, 36 weeks or less, 48 weeks or less, 12 months or less, 16 months or less, 20 months or less, or 24 months or less from starting treatment.

In additional embodiments, treatment with an effective amount of an anti-CTGF antibody, without the concomitant use of pirfenidone and/or nintedanib, is sufficient to produce a reduction, stabilization, or reversal of at least one or more of the following pulmonary radiographic parameters compared to a baseline measurement or historic controls: (i) degree of ground glass opacities; (ii) degree of parenchymal fibrosis (reticular opacities); and (iii) degree of honeycomb appearance of pulmonary architecture. Typically, these pulmonary radiographic parameters are evaluated by HRCT scans. For example, see Kim et al. Clin Exp Rheumatol. (2010) 28(5 Suppl 62):S26-S35; Kim et al. Eur Radiol (2011) 21: 2455-2465. As used herein, “stabilization” means the pulmonary radiographic parameter is substantially unchanged from baseline, i.e., within the error of measurement for the particular technique. As used herein, a “reduction” in a pulmonary radiographic parameter means a lessening of the severity of the parameter. Reductions of <−2%, i.e., more negative, in a pulmonary radiographic parameter compared to baseline are categorized as “reversals.”

In some embodiments, a reduction of at least 1%, 2%, 3%, 4%, 5%, 6%, 7%, 8%, 9%, 10%, 15%, 20%, 25%, 30%, 35%, 40%, 45%, or 50% is achieved in at least one pulmonary radiographic parameter compared to baseline or historic controls. For example, treatment with an effective amount of an anti-CTGF antibody, without the concomitant use of pirfenidone and/or nintedanib, reduces the pulmonary radiographic parameter ground glass opacities, parenchymal fibrosis or honey comb appearance by at least 2% compared to a baseline measurement resulting in a reversal of the pulmonary radiographic parameter. In further embodiments, the reduction, stabilization, or reversal in one or more pulmonary radiographic parameters is achieved in 3 weeks or less, 6 weeks or less, 9 weeks or less, 12 weeks or less, 18 weeks or less, 24 weeks or less, 36 weeks or less, 48 weeks or less, 12 months or less, 16 months or less, 20 months or less, or 24 months or less from starting treatment. Reductions in pulmonary radiographic parameters can also be measured serially, e.g., a comparison of HRCT scans at Weeks 24 and 48 compared to baseline may show an initial stabilization at Week 24 that continues to a reversal of the pulmonary radiographic parameter at Week 48.

In some embodiments, treatment with an effective amount of an anti-CTGF antibody, without the concomitant use of pirfenidone and/or nintedanib, is sufficient to produce an extension in the median progression-free survival or median overall survival of IPF subjects compared to historic controls, i.e., placebo treated. In some embodiments, the extension in median progression-free survival or median overall survival is at least two weeks, 1 month, 2 months, 3 months, 4 months, 5 months, 6 months, 7 months, 8 months, 10 months, 12 months, 14 months, 16 months, 18 months, 20 months, 24 months, 28 months, 32 months, 36 months, 40 months, or 48 months beyond the median progression-free survival or median overall survival of historic controls, i.e., placebo treated IPF patients. In particular embodiments, treatment with an effective amount of an anti-CTGF antibody, without the concomitant use of pirfenidone and/or nintedanib, produces a 5-year survival rate of at least 30%, 35%, 40%, 45%, or 50%.

In further embodiments, treatment with an effective amount of an anti-CTGF antibody, without the concomitant use of pirfenidone and/or nintedanib, is sufficient to decrease the risk of death due to IPF. In some embodiments, treatment with an effective amount of an anti-CTGF antibody, without the concomitant use of pirfenidone and/or nintedanib, reduces the 1-year risk, 2-year risk, 3-year risk, 4-year risk, 5-year risk, or 10-year risk of death by at least 5%, 10%, 15% , 20%, 25%, 30%, 35%, 40%, 50%, 60%, 70%, 80%, or 90% compared to subjects treated with historic controls, i.e., placebo treated.

In some embodiments, treatment with an effective amount of an anti-CTGF antibody, without the concomitant use of pirfenidone and/or nintedanib, is sufficient to produce one or more of the following: (i) the prevention of a worsening of dyspnea; (ii) the prevention of the development of new dyspnea; (iii) the reduction in the frequency or intensity of coughing; (iv) the prevention of a worsening of hypoxemia; (v) the reduction in the number or severity of acute exacerbations of IPF; (vi) the reduction in the number of respiratory-related hospital admissions; (vii) the reduction in the need for supplemental oxygen; (viii) the reduction in days of disability; or (ix) the improvement in the assessment of health-related quality of life (QoL). In particular embodiments, treatment with an effective amount of the anti-CTGF antibody without the concomitant use of pirfenidone and/or nintedanib reduces the frequency or intensity of coughing, reduces the number or severity of acute exacerbations of IPF, reduces the number of respiratory-related hospital admissions, reduces the need for supplemental oxygen and/or reduces the number of days of disability by at least 5%, 10%, 15%, 20%, 25%, 30%, 35%, 40%, 45%, 50%, 55%, 60%, 65%, 70%, 75%, 80%, 85%, 90%, or 95% compared to a baseline assessment or compared to subjects treated with historic controls, i.e., placebo treated.

By using the term “isolated” to describe an isolated antibody, antibody fragment, or antibody mimetic, it is intended that the molecule is not in its natural milieu. No particular level of purification is required. Recombinantly produced molecules are considered isolated for purposes of the invention, as are native molecules, e.g., polyclonal antibodies, which have been separated, fractionated, or partially or substantially purified by any suitable technique.

As used herein, “connective tissue growth factor” and “CTGF” refer to a matricellular protein belonging to a family of proteins identified as CCN proteins (Cysteine-rich 61 (Cyr61), Connective tissue growth factor (CTGF), Nephroblastoma overexpressed (Nov)). This family contains six distinct members (CYR61 (CCN1), CTGF (CCN2), NOV (CCN3), WISP-1(wnt-1 inducible secreted protein-1, CCN4), WISP-2 (CCNS), and WISP-3 (CCN6)) that share a high degree of amino acid sequence homology. (See, e.g., O'Brian et al. Mol Cell Biol (1990) 10:3569-3577; Joliot et al. Mol Cell Biol (1992) 12:10-21; Ryseck et al. Cell Growth and Diff (1991) 2:225-233; Simmons et al. Proc Natl Acad Sci USA (1989) 86:1178-1182; Pennica et al. Proc Natl Acad Sci USA, (1998) 95:14717-14722; and Zhang et al. Mol Cell Biol (1998) 18:6131-6141.)

CTGF may also be referred to within the art as “hypertrophic chondrocyte-specific protein 24,” “insulin-like growth factor-binding protein,” and “CCN2.” “CTGF” further refers to a substantially purified CTGF derived from any species, particularly a mammalian species, including rat, rabbit, bovine, ovine, porcine, murine, equine, and hominid, preferably the human species, and from any source, whether natural, synthetic, semi-synthetic, or recombinant.

### Antibodies

The term “antibody” is used in the broadest sense and specifically covers monoclonal antibodies (including full length monoclonal antibodies), polyclonal antibodies, multispecific antibodies (e.g., bispecific antibodies), and antibody fragments, so long as they exhibit the desired biological activity, and antibody mimetics.

The term “monoclonal antibody” as used herein refers to an antibody obtained from a population of substantially homogeneous antibodies, i.e., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. In certain embodiments, such a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody of this invention. In contrast to polyclonal antibody preparations, which typically include different antibodies directed against different determinants (epitopes), each monoclonal antibody of a monoclonal antibody preparation is directed against a single determinant on an antigen.

The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method (e.g., Kohler and Milstein, Nature, 256:495-97 (1975); Harlow et al., Antibodies: A Laboratory Manual (Cold Spring Harbor Laboratory Press, 2nd ed. 1988); recombinant DNA methods (see, e.g., U.S. Pat. No. 4,816,567); phage-display technologies (see, e.g., Clackson et al., Nature, (1991)352: 624-628; Marks et al., J Mol Biol (1992) 222: 581-597; and Lee et al., J Immunol Methods (2004) 284(1-2): 119-132), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc Natl Acad Sci USA (1993) 90: 2551; U.S. Pat. Nos. 5,545,807; 5,545,806; 5,569,825; 5,625,126; 5,633,425; and 5,661,016).

Monoclonal antibodies specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass (see, e.g., U.S. Pat. No. 4,816,567; and Morrison et al., Proc Natl Acad Sci USA (1984) 81:6851-6855).

“Humanized” forms of non-human (e.g., murine) antibodies are chimeric antibodies that contain minimal sequence derived from non-human immunoglobulin. In some embodiments, a humanized antibody is a human immunoglobulin (recipient antibody) in which residues from a one or more hypervariable regions (HVRs) of the recipient are replaced by residues from one or more HVRs of a non-human species (donor antibody) such as mouse, rat, rabbit, or nonhuman primate having the desired specificity, affinity, and/or capacity. For further details, see, e.g., Jones et al., Nature (1986) 321:522-525; Riechmann et al., Nature (1988);\ 332:323-329; and U.S. Pat. Nos. 6,982,321 and 7,087,409.

A “human antibody” is one which possesses an amino acid sequence which corresponds to that of an antibody produced by a human and/or has been made using any of the techniques for making human antibodies (see e.g., Hoogenboom and Winter, J Mol Biol, (1991) 227:381; Marks et al., J Mol Biol, (1991) 222:581; Boerner et al., J Immunol, (1991)47(1):86-95; Li et al., Proc Natl Acad Sci USA, (2006) 103:3557-3562 and U.S. Pat. Nos. 6,075,181 and 6,150,584).

A “naked antibody” for the purposes herein is an antibody that is not conjugated to a cytotoxic moiety or radiolabel. In some embodiments, the anti-CTGF antibody is a naked antibody.

The anti-CTGF antibodies that are suitable for the claimed use may be specific for CTGF endogenous to the species of the subject to be treated or may be cross-reactive with CTGF from one or more other species. In some embodiments, the antibody for use in the present methods is obtained from the same species as the subject in need. In other embodiments, the antibody is a chimeric antibody wherein the constant domains are obtained from the same species as the subject in need and the variable domains are obtained from another species. For example, in treating a human subject the antibody for use in the present methods may be a chimeric antibody having constant domains that are human in origin and variable domains that are mouse in origin. In preferred embodiments, the antibody for use in the present methods binds specifically to the CTGF endogenous to the species of the subject in need. Thus, in certain embodiments, the antibody is a human or humanized antibody, particularly a monoclonal antibody, that specifically binds human CTGF (GenBank Accession No. NP_001892).

Exemplary antibodies for use in the IPF treatment methods of the present invention are described, e.g., in U.S. Pat. No. 5,408,040; PCT/US1998/016423; PCT/US1999/029652 and International Publication No. WO 99/33878. Preferably, the anti-CTGF antibody for use in the IPF treatment method is a monoclonal antibody. Preferably the antibody is a neutralizing antibody. In particular embodiments, the antibody is the antibody described and claimed in U.S. Pat. Nos. 7,405,274 and 7,871,617. In some embodiments, the antibody for treatment of IPF has the amino acid sequence of the antibody produced by the cell line identified by ATCC Accession No. PTA-6006. In other embodiments, the antibody binds to CTGF competitively with an antibody produced by ATCC Accession No. PTA-6006. In further embodiments, the antibody binds to the same epitope as the antibody produced by ATCC Accession No. PTA-6006. A particular antibody for use in the IPF treatment methods is CLN1 or mAb1 as described in U.S. Pat. No. 7,405,274, or an antibody substantially equivalent thereto or derived therefrom. In some embodiments, the anti-CTGF antibody is CLN1, an antibody identical to the antibody produced by the cell line identified by ATCC Accession No. PTA-6006 that is encompassed by the claims of U.S. Pat. Nos. 7,405,274 and 7,871,617. In some embodiments the anti-CTGF antibody is pamrevlumab (CAS Registry Number 946415-13-0). Pamrevlumab is also known as FG-3019.

As referred to herein, the phrase “an antibody that specifically binds to CTGF” includes any antibody that binds to CTGF with high affinity. Affinity can be calculated from the following equation:

$$Affinity = {K}_{a} = \frac{[ Ab \cdot Ag ]}{[ Ab ] [ Ag ]} = \frac{1}{{K}_{d}}$$

where [Ab] is the concentration of the free antigen binding site on the antibody, [Ag] is the concentration of the free antigen, [Ab·Ag] is the concentration of occupied antigen binding sites, Kₐ is the association constant of the complex of antigen with antigen binding site, and K_{d} is the dissociation constant of the complex. A high-affinity antibody typically has an affinity at least on the order of 10⁸ M⁻¹, 10⁹ M⁻¹ or 10¹⁰ M⁻¹. In particular embodiments, an antibody for use in the present methods will have a binding affinity for CTGF between of 10⁸ M⁻¹ and 10¹⁰ M ⁻¹, between 10⁸ M⁻¹ and 10⁹ M⁻¹ or between 10⁹M⁻¹ and 10¹⁰ M⁻¹. In some embodiments the high-affinity antibody has an affinity of about 10⁸ M⁻¹, 10⁹ M⁻¹ or 10¹⁰ M⁻¹.

“Antibody fragments” comprise a functional fragment or portion of an intact antibody, preferably comprising an antigen binding region thereof A functional fragment of an antibody will be a fragment with similar (not necessarily identical) specificity and affinity to the antibody which it is derived. Non-limiting examples of antibody fragments include Fab, F(ab′)₂, and Fv fragments that can be produced through enzymatic digestion of whole antibodies, e.g., digestion with papain, to produce Fab fragments. Other non-limiting examples include engineered antibody fragments such as diabodies (Holliger P et al. Proc Natl Acad Sci USA. (1993), 90: 6444-6448); linear antibodies (Zapata et al. Protein Eng (1995) 8(10):1057-1062); single-chain antibody molecules (Bird K D et al. Science (1988), 242: 423-426); single domain antibodies, also known as nanobodies (Ghahoudi M A et al. FEBS Lett (1997) 414: 521-526); domain antibodies (Ward E S et al. Nature (1989) 341: 544-546); and multispecific antibodies formed from antibody fragments.

### Antibody Mimetics

Antibody mimetics are proteins, typically in the range of 3-25 kD, that are designed to bind an antigen with high specificity and affinity like an antibody, but are structurally unrelated to antibodies. Frequently, antibody mimetics are based on a structural motif or scaffold that can be found as a single or repeated domain from a larger biomolecule. Examples of domain-derived antibody mimetics include AdNectins that utilize the 10th fibronectin III domain (Lipovšek D. Protein Eng Des Sel, (2010) 24:3-9); Affibodies that utilize the Z domain of staphylococcal protein A (Nord K et al. Nat Biotechnol, (1997) 15: 772-777), and DARPins that utilize the consensus ankyrin repeat domain (Amstutz P. Protein Eng Des Sel. (2006) 19:219-229). Alternatively, antibody mimetics can also be based on the entire structure of a smaller biomolecule, such as Anticalins that utilize the lipocalin structure (Beste G et al. Proc Natl Acad Sci USA. (1999) 5:1898-1903). In some embodiments, the anti-CTGF antibody is an antibody mimetic.

### Pharmaceutical Compositions

The anti-CTGF antibodies, including antibody fragments and antibody mimetics, used in the claimed methods of the present invention can be delivered directly or in pharmaceutical compositions containing carriers and/or excipients, as is well known in the art. The anti-CTGF antibodies may be administered intravenously as a bolus or by continuous infusion over a period of time. Alternately, the anti-CTGF antibodies may be administered by intramuscular, subcutaneous, intradermal, subdermal or intraperitoneal injection, topical administration, or by inhalation. The route of administration may influence the type and composition of the formulation used in the anti-CTGF antibody preparation. Pharmaceutical compositions of particular interest include compositions suitable for injectable use and compositions suitable for nebulization or aerosolization.

The composition can be a liquid solution, suspension, emulsion, tablet, pill, capsule, sustained release formulation, powder, or lyophilized cake. Injectable forms include sterile aqueous solutions, dispersions and sterile powders for the extemporaneous preparation of sterile injectable solutions or dispersions.

Anti-CTGF antibody formulations for use in accordance with the present invention may be prepared by mixing an anti-CTGF antibody with pharmaceutically acceptable carriers, excipients or stabilizers that are nontoxic to subjects at the dosages and concentrations employed. Anti-CTGF antibody formulations may include buffers such as phosphate, citrate, and other organic acids; antioxidants including ascorbic acid and methionine; preservatives such as octadecyldimethylbenzyl ammonium chloride, hexamethonium chloride, benzalkonium chloride, benzethonium chloride, phenol, or benzyl alcohol; alkyl parabens including methyl or propyl paraben, catechol, resorcinol, cyclohexanol, 3-pentanol, and m-cresol; carriers; hydrophilic polymers such as polyvinylpyrrolidone; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes; and/or non-ionic surfactants or polyethylene glycol.

In particular, anti-CTGF antibody formulations may further comprise low molecular weight polypeptides; carriers such as serum albumin, gelatin, or immunoglobulins; and amino acids such as glycine, glutamine, asparagine, histidine, arginine, or lysine. The anti-CTGF antibody formulations can be lyophilized as described in PCT/US1996/012251. Additionally, sustained-release preparations may also be prepared. Frequently, polymers such as poly(lactic acid), poly(glycolic acid), or copolymers thereof serve as controlled/sustained release matrices, in addition to others well known in the art.

Numerous other pharmaceutically acceptable carriers, excipients, and stabilizers are available in the art, some of which are listed in various pharmacopoeias, e.g., US Pharmacopeia, Japanese Pharmacopeia, European Pharmacopeia, and British Pharmacopeia. Other sources include Gennaro, ed. (2000) Remington's Pharmaceutical Sciences, supra; and Goodman and Gilman's The Pharmacological Basis of Therapeutics, 10^{th} Ed. (2001), Hardman, Limbird, and Gilman, eds. MacGraw Hill Intl.; the Inactive Ingredient Search database maintained by the FDA and the Handbook of Pharmaceutical Additives, ed. Ash, Synapse Information Resources, Inc., 3rd Ed. 2007.

Compositions formulated for parenteral administration by injection are usually sterile and can be presented in unit dosage forms, e.g., in ampoules, syringes, injection pens, or in multi-dose containers, the latter usually containing a preservative. In certain instances, such as with a lyophilized product or a concentrate, the parenteral formulation would be reconstituted or diluted prior to administration.

The anti-CTGF antibodies can be supplied or administered at any desired concentration. In some embodiments, the anti-CTGF antibody concentration is at least 1 mg/ml, 5 mg/ml, 10 mg/ml, 20 mg/ml, 25 mg/ml, 50 mg/ml, 75 mg/ml, 100 mg/ml, 125 mg/ml, 150 mg/ml, or 200 mg/ml. In other embodiments, the anti-CTGF antibody concentration is no more than about 5 mg/ml, 10 mg/ml, 20 mg/ml, 25 mg/ml, 50 mg/ml, 75 mg/ml, 100 mg/ml, 125 mg/ml, 150 mg/ml, 200 mg/ml, 250 mg/ml, or 300 mg/ml. In further embodiments, the anti-CTGF antibody concentration is between 5 mg/ml to 20 mg/ml, 20 mg/ml to 50 mg/ml, 50 mg/ml to 100 mg/ml, 100 mg/ml to 200 mg/ml, or 200 mg/ml to 300 mg/ml.

### Dosage

A therapeutically effective amount of an anti-CTGF antibody can be administered in one or more administrations, applications or dosages. The skilled artisan will appreciate that certain factors may influence the dosage and timing required to effectively treat a subject, including but not limited to the severity or extent of the disease, the administration route, previous treatments, concurrent medications, performance status, weight, gender, race or ethnicity, and/or age of the subject.

In some embodiments, the method for treating IPF in a subject in need thereof comprises administering at least 0.5 g, at least 1.0 g, at least 1.5 g, at least 2.0 g, at least 2.5 g, or at least 3.0 g of an anti-CTGF antibody per a one, two, or three week period, optionally, in combination with at least one additional IPF therapeutic agent, provided that the additional IPF therapeutic agent is not pirfenidone and/or nintedanib. In specific embodiments, the anti-CTGF antibody is administered at a dose of about 1.05 g or about 2.1 g every three weeks, based on a 70 kg standard man, optionally, in combination with at least one additional IPF therapeutic agent, provided that the additional IPF therapeutic agent is not pirfenidone and/or nintedanib.

In a further embodiment, the method for treating IPF in a subject in need thereof comprises administering at least 10 mg/kg, 15 mg/kg, 20 mg/kg, 25 mg/kg, 30 mg/kg, 35 mg/kg, 40 mg/kg, 50 mg/kg, or 60 mg/kg of an anti-CTGF antibody per a one, two, or three week period, optionally, in combination with at least one additional IPF therapeutic agent, provided that the additional IPF therapeutic agent is not pirfenidone and/or nintedanib. In particular embodiments, the anti-CTGF antibody is administered at a dose of about 15 mg/kg, about 30 mg/kg or about 35 mg/kg every three weeks optionally, in combination with at least one additional IPF therapeutic agent, provided that the additional IPF therapeutic agent is not pirfenidone and/or nintedanib. In other embodiments, the anti-CTGF antibody is administered at a dose of about 30 mg/kg or 35 mg/kg every two weeks, optionally, in combination with at least one additional IPF therapeutic agent, provided that the additional IPF therapeutic agent is not pirfenidone and/or nintedanib.

In some embodiments, a method for treating IPF presented herein involves the administration to a subject in need thereof of an anti-CTGF antibody at a dose that achieves a target plasma concentration of the anti-CTGF antibody in the subject. In some embodiments, the target plasma concentration of an anti-CTGF antibody is a maximum antibody concentration (Cₘₐₓ) in the plasma, typically seen immediately after i.v. administration to the subject. In particular embodiments, the method for treating IPF achieves a Cₘₐₓ the antibody of at least 10 μg/ml, 50 μg/ml, 100 μg/mL, 125 μg/mL, 150 μg/mL, 200 μg/mL, 300 μg/mL, or 400 μg/mL.

In other embodiments, the target plasma concentration is a minimum antibody concentration (Cₘᵢₙ) in the plasma, also known as a trough antibody concentration, which is typically measured immediately before a subsequent antibody administration to the subject. In some embodiments, the Cₘᵢₙ plasma concentration of the anti-CTGF antibody is at least 0.1 μg/ml, 1.0 μg/ml, 5μg/ml, 10 μg/mL, 20 μg/ml, 30 μg/ml, 40 μg/ml, 50 μg/ml, 60 μg/ml, 70 μg/ml, 80 μg/ml, 90 μg/ml, 100 μg/ml, 125 μg/ml, 150 μg/ml, 200 μg/ml, 300 μg/ml, or 400 μg/ml. In further embodiments, Cₘᵢₙ is measured for a treatment cycle of about 2, 3, 4, 5, 6, 7, 8, 9, 10, 11, 12, 13, 14, 15, 16, 17, 18, 19, 20, 21, 22, 23, 24, or 28 days. In a particular embodiment, the Cₘᵢₙ is at least 150 μg/mL when measured at about 21 days after administration of an anti-CTGF antibody dose.

In further embodiments, a method for treating IPF in a subject in need thereof comprises the administration of an anti-CTGF antibody at a dose that achieves a target antibody exposure (area under the curve, AUC) over a specific time period. Typically, AUC is expressed as μg*h/ml. In some embodiments, a method for treating IPF in a subject in need thereof comprises the administration to a subject an anti-CTGF antibody at a dose that achieves an AUC in plasma of at least 1,000 μg*h/ml, 10,000 μg*h/ml, 25,000 μg*h/ml, 50,000 μg*h/ml, 60,000 μg*h/ml, 80,000 μg*h/ml, 100,000 μg*h/ml, 120,000 μg*h/ml, or 140,000 μg*h/ml. In some embodiments, the AUC is calculated from about 0-4 days, 0-5 days, 0-6 days, 0-7 days, 0-8 days, 0-9 days, 0-10 days, 0-11 days, 0-12 days, 0-13 days, 0-14 days, 0-16 days, 0-18 days 0-21 days, or 0-28 days. In a particular embodiment, the AUC is at least 1,000 μg*h/ml when measured from 0-21 days post-administration (AUC0-21).

In some embodiments, the patient is treated for a minimum of 2 weeks, 3 weeks, 4 weeks, 6 weeks, 9 weeks, 12 weeks, 15 weeks, 18 weeks, 21 weeks, 24 weeks, 27 weeks, 30 weeks, 36 weeks, 40 weeks, 48 weeks, 1 year, or 2 years. In other embodiments, the patient is treated every 1 week, 2 weeks, 3 weeks, 4 weeks, 5 weeks, 6 weeks, 8 weeks, 10 weeks, or 12 weeks as indicated by the patient's healthcare practitioner. In additional embodiments, the patient is treated for a maximum of 6 weeks, 9 weeks, 12 weeks, 15 weeks, 18 weeks, 21 weeks, 24 weeks, 27 weeks, 30 weeks, 36 weeks, 40 weeks, 48 weeks, 1 year, 2 years, 3 years, 4 years, or 5 years. In further embodiments, the treatment duration is between 1 week to 24 weeks, 24 weeks to 48 weeks, 48 weeks to 2 years, 3 weeks to 2 years or 3 weeks to 3 years.

In some embodiments, the anti-CTGF antibody or a pharmaceutical composition comprising the antibody is administered through a bolus injection intravenously. In other embodiments, the anti-CTGF antibody is administered as an infusion that can be for a duration of not less than 10 minutes, 20 minutes, 30 minutes, 1 hour, 2 hours, 4 hours, or 8 hours. In further embodiments, the anti-CTGF antibody is administered subcutaneously in a concentrated form. In other embodiments, the anti-CTGF antibody is administered as an aerosolized powder or a nebulized solution for inhalation.

In specific embodiments, a method for treating IPF presented herein involves the administration to a subject in need thereof of an anti-CTGF antibody or a pharmaceutical composition thereof, without the concomitant administration of pirfenidone and/or nintedanib, at a dosage and/or a frequency of administration that produces a functional outcome, e.g., stabilization or reversal of decline in FVC. In other embodiments, a method for treating IPF presented herein involves the administration to a subject in need thereof of an anti-CTGF antibody or a pharmaceutical composition thereof, without the concomitant administration of pirfenidone and/or nintedanib, at a dosage and/or a frequency of administration that produces an outcome that can be imaged such as a reduction or reversal in a pulmonary radiographic parameter or inflammation, as assessed by HRCT scan, chest x-ray, histopathologically, or another modality.

### Subjects Suitable for Treatment

The methods of the invention are appropriate for the treatment of subjects diagnosed with IPF or UIP using any method recognized in the art including HRCT, chest x-rays, transbronchial biopsy and/or surgical lung biopsy. The methods of the invention are also appropriate for the treatment of subjects suspected of having IPF based on the presence of one or more characteristics known in the art to be indicative of the presence of IPF. These characteristics include progressive dyspnea and cough, bibasilar inspiratory crackles, digital clubbing, and non-specific bilateral, reticular infiltrates in the periphery of the lower lung zones visible on a chest radiograph. Further characteristics indicative of IPF include reduced lung volumes, a proportionate reduction in the pulmonary diffusing capacity or a normal to increased FEV1/FVC ratio demonstrated in pulmonary function tests. Other characteristics indicative of IPF include resting arterial blood hypoxemia, oxyhemoglobin desaturation, or an increased alveolar-arterial oxygen pressure difference, any of which may worsen with exercise. Additional abnormalities during exercise that may indicate the presence of IPF include reduced peak oxygen consumption, diminished ventilatory reserve, high-frequency/low tidal volume breathing pattern, and high submaximal ventilation related in part to elevated physiologic dead space and arterial desaturation. A further characteristic indicative for IPF is the presence of pulmonary hypertension.

In some embodiments, one or more of the following pulmonary function parameters are used to select subjects for therapy with an anti-CTGF antibody or to monitor response to anti-CTGF antibody therapy: VC, FVC, FVCPP, RV, FEV, PEFR, IRV, FIF, FRC, IC, TLC, ERV, TV, or MVV. In particular embodiments, the pulmonary function parameters TLC, FVC, and FVCPP are used to select and/or monitor subjects.

Subjects that are particularly suited for treatment with the method of the invention are those that have a FVCPP value of at least 35%, 40%, 45%, 50%, 55%, 60%, 63%, 65%, 70%, 75%, 80%, 85%, 90%, or 95% of a normal person of similarly matched race or ethnicity, gender, age, height and weight. In other embodiments, subjects suitable for treatment with the method of the invention are those that have a FVCPP value of not more than 40%, 45%, 50%, 55%, 60%, 65%, 70%, 75%, 80%, 85%, 90%, or 95%. In further embodiments, subjects suitable for treatment have a FVCPP value of between 40% to 95%, 50% to 90%, 55% to 85%, 60% to 80%, 55% to 80%, 60% to 70%, 70% to 90%, 60% to 90%, or 70% to 95%. In particular embodiments, the subjects have a FVCPP value of about 55%-85%.

Additional subjects that are particularly suited to treatment with an anti-CTGF antibody, without the concomitant use of pirfenidone and/or nintedanib are those that have at least 40%, 45%, 50%, 55%, 60%, 65%, 70%, 75%, 80%, 85%, 90%, or 95% of the predicted TLC of a normal person of similarly matched race or ethnicity, gender, age, height and weight. In other embodiments, subjects suitable for treatment with the method of the invention are those that have a not more than 40%, 45%, 50%, 55%, 60%, 65%, 70%, 75%, 80%, 85%, 90%, or 95% of the predicted TLC. In further embodiments, subjects suitable for treatment have between 40% to 95%, 45% to 90%, 50% to 85%, 55% to 85%, 50% to 70%, 60% to 80%, or 70% to 95% of the predicted TLC.

Further subjects that are particularly suited to treatment with an anti-CTGF antibody, without the concomitant use of pirfenidone and/or nintedanib are those that have at least 40%, 45%, 50%, 55%, 60%, 65%, 70%, 75%, 80%, 85%, 90%, or 95% of the predicted FEV1 of a normal person of similarly matched race or ethnicity, gender, age, height and weight. In other embodiments, subjects suitable for treatment with the method of the invention are those that have a not more than 40%, 45%, 50%, 55%, 60%, 65%, 70%, 75%, 80%, 85%, 90%, or 95% of the predicted FEV1. In further embodiments, subjects suitable for treatment have between 40% to 95%, 45% to 90%, 50% to 85%, 55% to 85%, 50% to 70%, 60% to 80%, or 70% to 95% of the predicted FEV1.

In further embodiments, the subjects suitable for treatment with an anti-CTGF antibody, without the concomitant use of pirfenidone and/or nintedanib, have a pathologic rate of decline in one or more pulmonary function parameters of at least 5%, 10%, 15%, 20%, 30%, 40%, 50%, 60%, 70%, 80%, 90%, 100%, 125%, 150%, 200%, 300%, 400%, 500%, 600%, 700%, 800% or 1,000% over the expected rate of decline for a normal person of similarly matched race or ethnicity, gender, age, height and weight.

Subjects that are particularly suited for treatment with an anti-CTGF antibody, without the concomitant use of pirfenidone and/or nintedanib, further include those that have a DLCO % predicted value corrected for blood hemoglobin of at least 25%, 30%, 35%, 40%, 45%, 50%, 55%, 60%, 65%, 70%, 75%, 80%, 85%, 90%, or 95%. In other embodiments, subjects suitable for treatment with the method of the invention are those that have a DLCO % predicted value corrected for blood hemoglobin of at least 25%, but not more than 40%, 45%, 50%, 55%, 60%, 65%, 70%, 75%, 80%, 85%, 90%, or 95%. In further embodiments, subjects suitable for treatment have a DLCO % predicted value corrected for blood hemoglobin between 30% to 95%, 40% to 90%, 45% to 85%, 50% to 90% or 60% to 80%.

Additional subjects that are particularly suited for treatment with an anti-CTGF antibody, without the concomitant use of pirfenidone and/or nintedanib, are those that have a SaO₂ of at least 70%, 75%, 80%, 85%, 90%, 91%, 92%, 93%, 94%, 95%, 96%, 97%, 98%, or 99%. In other embodiments, subjects suitable for treatment with the method of the invention are those that have a SaO₂ of at least 70%, but not more than 80%, 85%, 90%, 91%, 92%, 93%, 94%, 95%, 96%, 97%, 98%, or 99%. In further embodiments, subjects suitable for treatment have a SaO₂ of between 70% to 95%, 70% to 99%, or 80% to 99%.

Other subjects that are particularly suited for treatment with an anti-CTGF antibody, without the concomitant use of pirfenidone and/or nintedanib, are those that have an [A-a] PO₂ of at least 10 mmHg, 20 mmHg, 30 mmHg, 40 mmHg, 50 mmHg, 75 mmHg, 100 mmHg, 125 mmHg, 150 mmHg, 175 mmHg, 200 mmHg, or 250 mmHg. In other embodiments, subjects suitable for treatment have a [A-a] PO₂ between 10 mmHg to 50 mmHg, 10 mmHg to 100 mmHg, 10 mmHg to 200 mmHg, 20 mmHg to 250 mmHg, 50 mmHg to 250 mmHg, or 100 mmHg to 250 mmHg.

Further subjects that are particularly suited to treatment with an anti-CTGF antibody, without the concomitant use of pirfenidone and/or nintedanib, are those subjects that are not more than 20 years old, 25 years old, 30 years old, 35 years old, 40 years old, 45 years old, 50 years old, 55 years old, 60 years old, 65 years old, 70 years old, 75 years old, 80 years old, 85 years old, or 90 years old. In other embodiments, subjects that are particularly suited to treatment with the method of the invention are those subjects that are not less than 20 years old, 25 years old, 30 years old, 35 years old, 40 years old, 45 years old, 50 years old, 55 years old, 60 years old, 65 years old, 70 years old, 75 years old, 80 years old, 85 years old, or 90 years old. In further embodiments, subjects that are particularly suited to treatment with the method of the invention are those subjects that are between 30 years old to 80 years old, 40 years old to 90 years old, 50 years old to 100 years old, or 55 years old to 95 years old.

The methods are also suitable for the treatment of subjects with IPF who were previously treated with conventional therapies and failed to respond or experienced unacceptable toxicities associated with these therapies, including pirfenidone monotherapy or nintedanib monotherapy, corticosteroids and/or immunosuppressive drugs. Pirfenidone usage is associated with gastrointestinal toxicities. In combined clinical studies the following toxicities were seen in pirfenidone treated patients: nausea, 36%; vomiting, 13%; abdominal pain 24%; and diarrhea 26% of anorexia. Another toxicity seen was skin rash seen in 30% of the combined patients.

Nintedanib associated toxicities include hepatic impairment, elevated liver enzymes and drug-induced liver injury that can lead to a fatal outcome. Patients at higher risk for elevated liver enzymes include female and Asians patients and those with a low body weight. Gastrointestinal toxicities are the most commonly seen toxicities with diarrhea being the most frequently reported event with 62% of patients in clinical trials experiencing this toxicity. Nausea, abdominal pain and vomiting are also common with 24%, 15% and 12%, respectively, of patients in clinical trials reported experiencing these adverse events. Arterial thromboembolic events have also been reported with 2.5% of patients in a clinical trial of nintedanib, of which the most common event was myocardial infarction.

Accordingly, subjects with known or suspected hepatic impairment, elevated risk of a cardiovascular event, including past myocardial infarction, photosensitivity, gastrointestinal sensitivities, etc., as well as those that have ceased treatment with pirfenidone and/or nintedanib because of their toxicities are candidates for treatment with an anti-CTGF antibody.

In some embodiments, the claimed method has an improved safety profile compared to treatment with pirfenidone and/or nintedanib. In further embodiments, the improved safety profile is a reduction, compared to subjects treated with pirfenidone and/or nintedanib, of experiencing hepatic impairment, including drug-induced liver damage; risk of a experiencing a cardiovascular event, including myocardial infarction; photosensitivity; or gastrointestinal toxicities, including nausea, diarrhea, dyspepsia, vomiting and anorexia. In further embodiments, treatment with an effective amount of an anti-CTGF antibody has a reduction in risk, compared to treatment with pirfenidone and/or nintedanib, of at least 5%, 10%, 20%, 30%, 40%, 50%, 60%, 70%, 80%, 90% or 95% of experiencing hepatic impairment, including drug-induced liver damage; risk of a experiencing a cardiovascular event, including myocardial infarction; photosensitivity; or gastrointestinal toxicities, including nausea, diarrhea, dyspepsia, abdominal pain, vomiting and anorexia.

The methods of the invention are additionally suitable for subjects who are at risk of developing IPF. Those at risk include former and current smokers; those of the male gender; those with an age of 60 years or more; those with gastroesophageal reflux disease or those with a genetic predisposition for developing IPF.

### Articles of Manufacture

The present compositions may, if desired, be presented in a pack or dispenser device containing one or more unit dosage forms containing the anti-CTGF antibody and additional therapies. Such a pack or device may, for example, comprise metal or plastic foil, glass and rubber stoppers, such as in vials, or syringes. The container holds or contains an anti-CTGF antibody composition that is effective for treating IPF and may have a sterile access port (for example the container may be an intravenous solution bag or a vial having a stopper pierceable by a hypodermic injection needle). The container holding the anti-CTGF antibody compositions may further be labeled for the treatment of IPF and may include instructions not to concomitantly administer pirfenidone and/or nintedanib with the anti-CTGF antibody. The pack or dispenser device may be accompanied by instructions for administration including specific guidance regarding dosing amounts for the anti-CTGF antibody and may also include instructions warning against the co-administration of not pirfenidone and/or nintedanib with the anti-CTGF antibody. Embodiments in which the anti-CTGF antibody and one or more additional therapies are packaged or are administered as fixed-dose combination form are specifically encompassed herein.

The article of manufacture may further comprise an additional container comprising a pharmaceutically acceptable diluent buffer, such as bacteriostatic water for injection (BWFI), phosphate-buffered saline, Ringer's solution, and/or dextrose solution. The article of manufacture may further include other materials desirable from a commercial and user standpoint, including other buffers, diluents, filters, needles, and syringes.

These and other embodiments of the present invention will readily occur to those of ordinary skill in the art in view of the disclosure herein.

### EXAMPLES

The invention will be further understood by reference to the following examples, which are intended to be purely exemplary of the invention. The present invention is not limited in scope by the exemplified embodiments, which are intended as illustrations of single aspects of the invention only. Any methods that are functionally equivalent are within the scope of the invention. Various modifications of the invention in addition to those described herein will become apparent to those skilled in the art from the foregoing description and accompanying figures. Such modifications are intended to fall within the scope of the appended claims.

### Example 1: Pamrevlumab in Combination with Pirfenidone and/or Nintedanib in a Mouse Radiation-Induced Lung Fibrosis Model

The objective of this study was to compare the activity of pamrevlumab monotherapy to either pirfenidone or nintedanib alone, and to evaluate the potential for enhanced activity of the combined agents. There are no mouse models of IPF that replicate all of the major aspects of the disease, but the radiation-induced lung fibrosis model (see, Bickelhaupt et al. J Natl Cancer Inst. (2017) 109(8) doi:10.1093/jnci/djw339) exhibits progressive pulmonary fibrosis and functional decline that allows assessment of true therapeutic intervention. In this study, mice received thoracic irradiation (RT), followed by the therapeutic administration of the agents at 16 weeks post-irradiation when lung remodeling was evident by HRCT. After 8 weeks of drug treatment, lung density and volume were assessed by HRCT, lung function was assessed by blood gas analysis, and mice were sacrificed for histologic and gene expression analysis.

C57B1/6 mice were irradiated with 14.5 Gy photons; full thorax, single dose. Treatment with therapeutic agent(s) (pamrevlumab, pirfenidone, and/or nintedanib) was initiated 16 weeks post-irradiation and continued for 8 weeks. Pamrevlumab (FG) was given IP, BIW, at 40 mg/kg; pirfenidone (PF) was given PO in chow, QD, at 300 mg/kg; nintedanib (ND) was given PO in chow, QD, at 100 mg/kg. Treatment with each agent alone, with combinations of two, and with a triple combination were carried out. Controls included non-irradiated mice, mice that were irradiated and not treated, and mice that were irradiated and given human IgG. Endpoint measurements were taken at 24 weeks for HRCT (lung structure), blood gas analysis (lung function), histology and gene expression analysis.

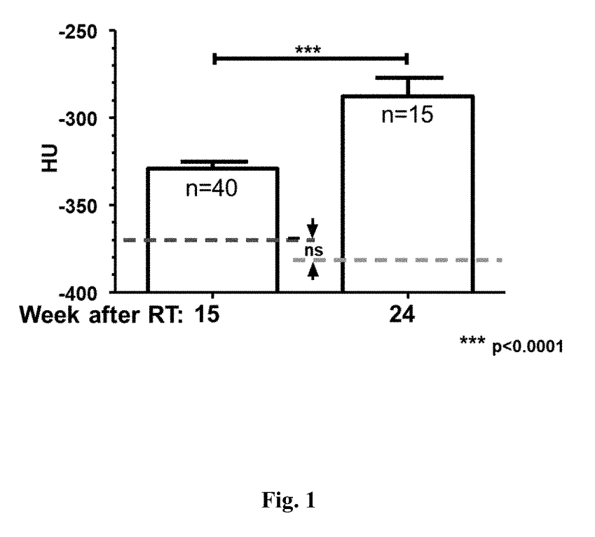

Lung density (Hounsfield Units) at 15 weeks and 24 weeks was increased in irradiated mice relative to non-irradiated mice (FIG. 1). Irradiated mice displayed increased lung density compared to non-irradiated mice at both time points. Lung density was significantly increased in the irradiated and not treated mice at week 24 compared to week 15. A small, statistically non-significant decrease of the lung density of non-irradiated mice over the same time period was observed.

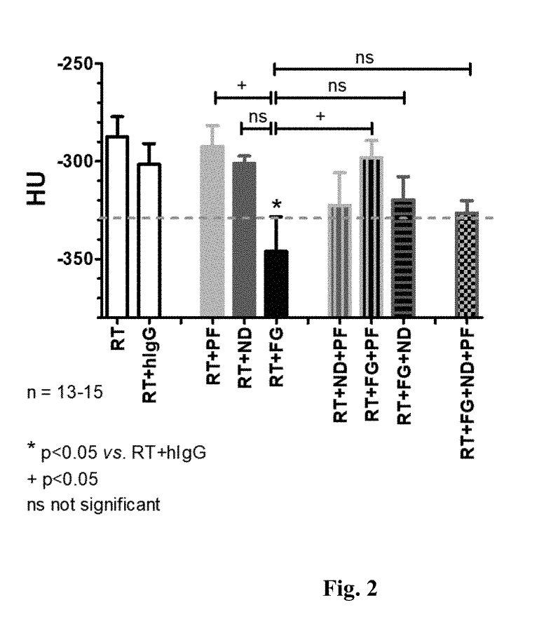

At 24 weeks, only pamrevlumab monotherapy (RT+FG) significantly inhibited the increase in radiation-induced lung density from baseline (dashed line), and was the only effective monotherapy (FIG. 2).

Combinations of pamrevlumab with either pirfenidone and/or nintedanib were less efficacious at inhibiting lung remodeling than pamrevlumab monotherapy, with the combination of pamrevlumab and pirfenidone being statistically significantly worse than pamrevlumab monotherapy. The data demonstrate that standard of care IPF drugs attenuate the benefit of pamrevlumab with respect to improvements in lung remodeling and lung density. Accordingly, subjects that have or are scheduled to receive treatment with an anti-CTGF antibody, e.g., pamrevlumab, should not be administered concomitantly pirfenidone and/or nintedanib.

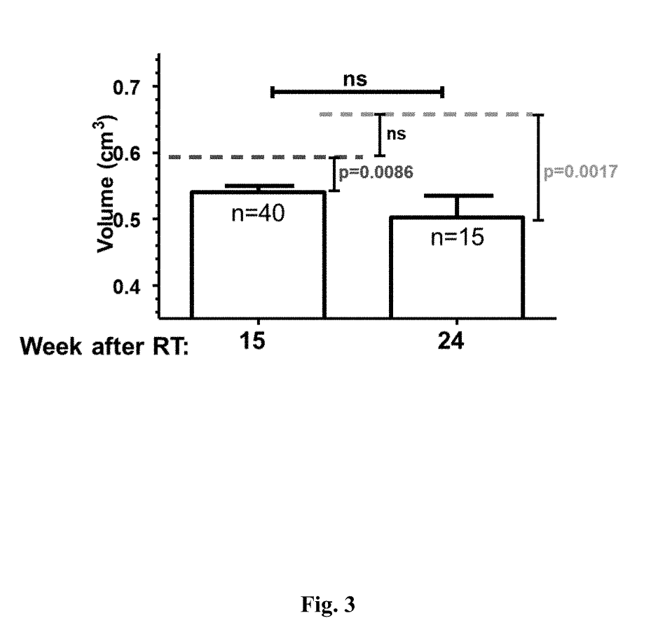

Lung volumes measured at 15 weeks were significantly decreased in irradiated mice relative to non-irradiated controls, p=0.0086 (FIG. 3). At 24 weeks, a small, non-significant increase in the lung volume of the non-irradiated control mice was observed. Lung volumes of irradiated and not treated mice were significantly reduced at 24 weeks compared to the non-irradiated controls, p=0.0017.

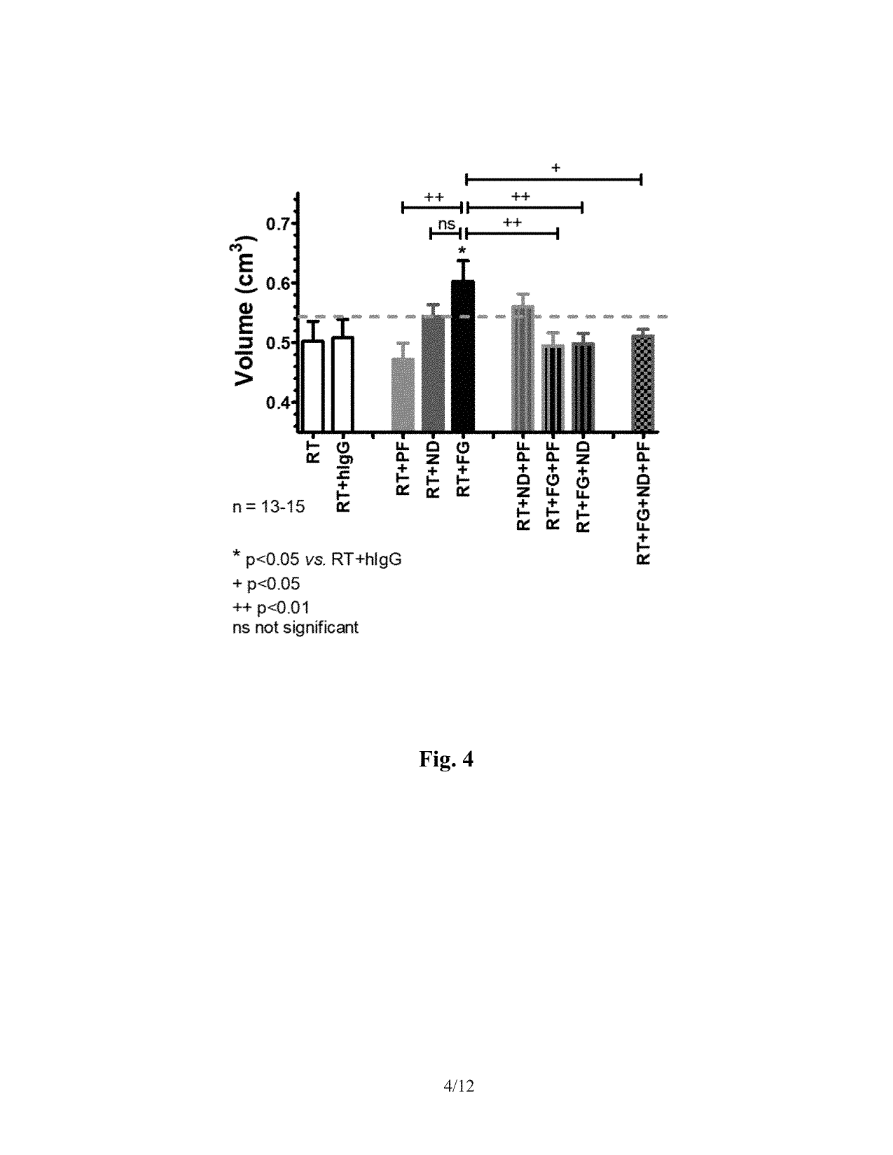

At 24 weeks, only pamrevlumab monotherapy (RT+FG) significantly inhibited radiation-induced lung volume decrease from baseline (dashed line), and was the only effective monotherapy to significantly improve lung volume (FIG. 4). Nintedanib monotherapy appeared to inhibit lung volume decrease from baseline, but the difference between nintedanib-treated and untreated irradiated control mice (RT) was not statistically different. Pamrevlumab monotherapy was statistically significantly different from pamrevlumab combined with other agents, demonstrating that the standard of care IPF drugs attenuated the benefits of pamrevlumab with respect to improvements in lung volume. These data again demonstrate that the administration of pirfenidone and/or nintedanib is contraindicated in subjects that are or will be treated with an anti-CTGF antibody, e.g., pamrevlumab.

The effect of the treatments on lung function was assessed by blood gas analysis (data not shown). Monotherapy with pamrevlumab or nintedanib normalized O₂ saturation compared to irradiation (RT) with no treatment. All combination therapies showed improved O₂ saturation compared to RT without treatment.

Microarray analysis of mRNA transcripts of numerous genes at 24 weeks after RT were carried out by standard techniques to compare the expression profiles of irradiated and treated animals to irradiated animals that were not further treated. The results of the later group, RT (control), were used to normalize the expression data from the other groups. Briefly, RNA from 4-7 animals per condition (RT (control), RT+hIgG, RT+FG, RT+ND, RT+PF, RT+FG+ND, RT+FG+PF, RT+ND+PF, RT+FG+ND+PF). Affymetrix 430 2.0 data was used. RT induced changes (≥1.5×, p<0.05) were filtered for treatment dependent correction (>50% correction, p<0.05). 80 RT-induced and 68 RT-suppressed transcripts were normalized at least 50% by one or more treatments. The resulting list was dominated by pamrevlumab-dependent normalization. Functional analysis indicated pamrevlumab treatment preferentially normalized RT genes known to be regulated by TGFβ and TNF.

The data demonstrate that pamrevlumab monotherapy normalized more fibrosis-related transcripts than other monotherapies. (See Table 1). Further, pirfenidone (PF) and (ND) monotherapies appeared to exacerbate RT-induced changes of several fibrosis-related transcripts. For instance, PF or ND monotherapy was associated with a further induction of expression of the Wisp 1 and Nt5e genes compared to RT treatment alone. In addition, combinations of pamrevlumab with pirfenidone and/or nintedanib often attenuated pamrevlumab monotherapy-normalization of gene expression demonstrating the ability of these agents to antagonize some of the homeostatic benefits of pamrevlumab. The gene expression data corroborate the lung density and lung volume results demonstrating that monotherapy with an anti-CTGF antibody is more efficacious than monotherapy with either pirfenidone and/or nintedanib. Further, the gene expression data also demonstrate that combining pirfenidone and/or nintedanib with pamrevlumab attenuates of the benefits of pamrevlumab monotherapy, i.e., normalization of gene expression seen with pamrevlumab monotherapy.

TABLE 1
Normalization of Gene Expression Levels in Irradiated
Lung Following Various Treatments

| | | % Normalization | | | | | | |
|---|---|---|---|---|---|---|---|---|
| | RT | | | | FG + | | | |
| Fibrosis | Fold | | FG + | FG + | ND + | | | ND + |
| Genes | Change | FG | ND | PF | PF | ND | PF | PF |
| Apln | 5.3 | 59% | 44% | 44% | 32% | 6% | 41% | 49% |
| Wisp1 | 11.9 | 54% | 57% | 43% | 43% | −18% | −78% | 20% |
| Wisp1 | 6.9 | 52% | 57% | 43% | 46% | −7% | −21% | 47% |
| Igfbp7 | 2.6 | 50% | 42% | 42% | 31% | 10% | −6% | 39% |
| Retnla | 2.0 | 43% | 53% | 65% | 56% | −9% | −7% | 9% |
| Ccr2 | 2.4 | 37% | 52% | −49% | 21% | 10% | −20% | −2% |
| Cdkn2a | 2.7 | 35% | 46% | 39% | 52% | 9% | −37% | 64% |
| Abca3 | 0.4 | 75% | 28% | 38% | 37% | −35% | −20% | 37% |
| Igfbp5 | 0.3 | 70% | 63% | 66% | 60% | 54% | 27% | 71% |
| Gdf2 | 0.4 | 68% | 53% | 40% | 42% | 33% | 10% | 40% |
| Cx3cl1 | 0.5 | 57% | 10% | 5% | −2% | −21% | −8% | −32% |
| Nt5e | 2.1 | 53% | −6% | 13% | −7% | −22% | −58% | −38% |
| Plat | 2.2 | 51% | 20% | −22% | 20% | −55% | 0% | 2% |

### Example 2: Quality of Life Improvement in IPF Patients Treated with Pamrevlumab

A double-blind, placebo-controlled Phase two study was conducted in which 103 patients, randomized (1:1) to receive pamrevlumab or placebo, were treated for 48 weeks. Patients' self-administered the Saint George's Respiratory Questionnaire (SGRQ) to assess changes in health-related quality of life parameters over the course of the study. This questionnaire was developed for patients with chronic airflow limitation and the results correlate well with established measures of symptom level, disease activity and disability. Patients completed the SGRQ on Day 1 (baseline) and every 12 weeks thereafter during the 48-week treatment period. The SGRQ comprises three domains (symptoms, activity, and impact) with the score for each domain ranging from 0 to 100, with higher scores indicating worse health-related quality of life (Jones P W, et al. Respir Med 1991; 85:Suppl B:25-31; Barr J T et al. Clin Ther 2000; 22:1121-45).

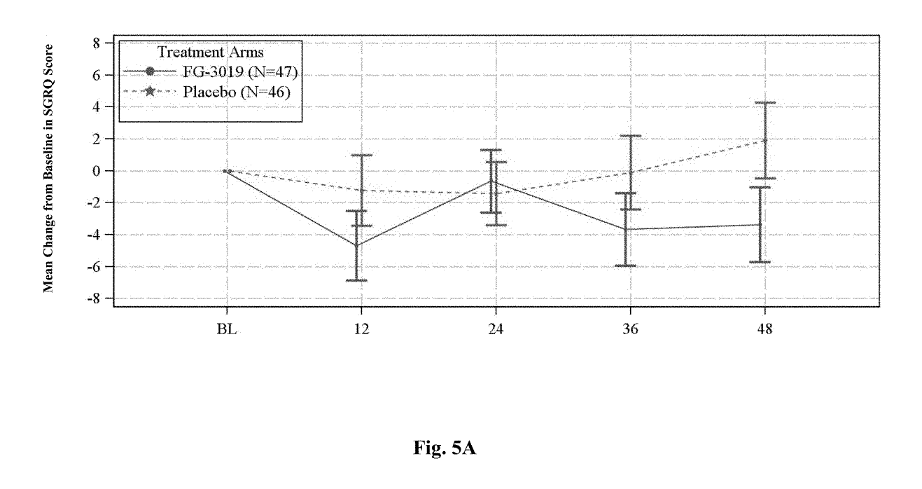

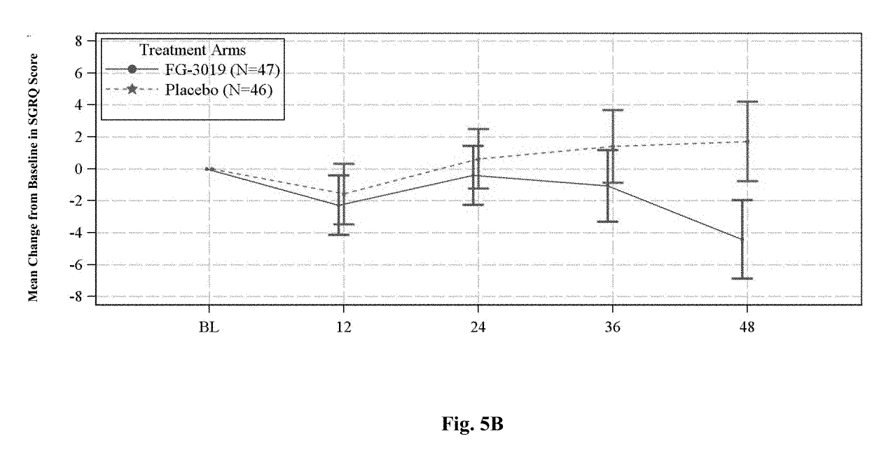

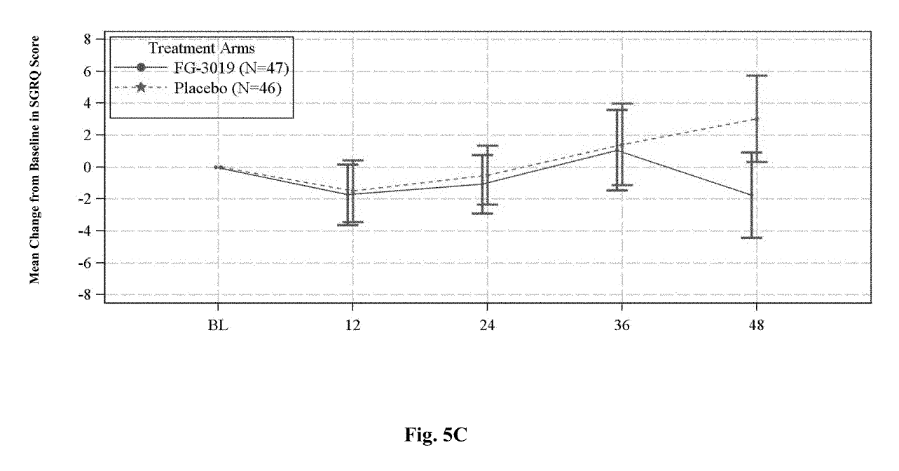

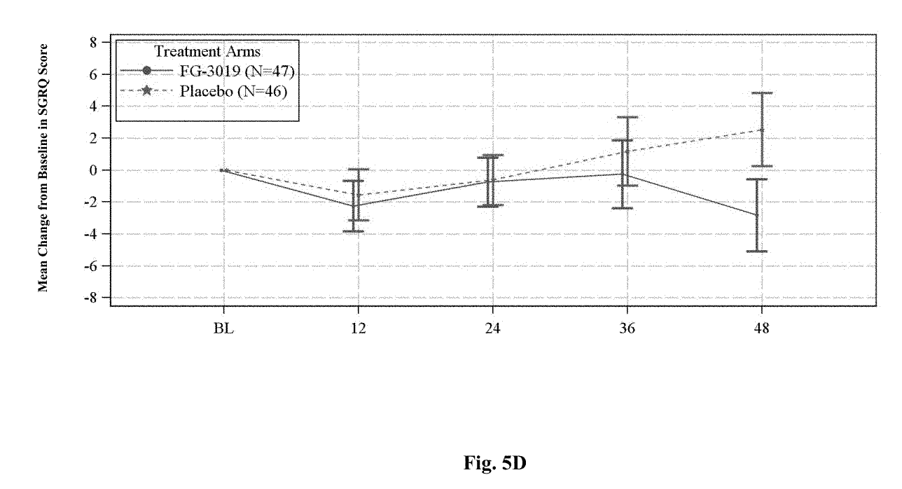

The SGRQ results showed improvement (lower values) for pamrevlumab-treated patients across all domains and total score compared with a worsening (higher values) of all domains and total score for placebo-treated patients (FIGS. 5A-5D). These findings demonstrate clinically meaningful improvement in the quality of life for patients suffering from IPF was achieved with treatment of an anti-CTGF antibody.

### Example 3: Effects of Pamrevlumab on Dyspnea

A sub-set of patients in the Phase two clinical trial described above in Example 2 (pamrevlumab n=22, placebo n=20), self-administered the University of California, San Diego—Shortness of Breath Questionnaire (UCSD-SOBQ) at the start of therapy (baseline) and then every 12 weeks while enrolled in the 48 week study. The UCSD-SOBQ has been validated as an acceptable measure to assess change in dyspnea over time in IPF (Swigris et al., Respir Med. 2012 October; 106(10): 1447-1455).

The questionnaire has 24 sections that assess dyspnea associated with activities of daily living (ADLs) in different lung disorders, including IPF. Twenty-one sections relate to the severity of dyspnea experienced during different daily activities, while three sections assess the limitations due to shortness of breath, fear of harm from overexertion and fear of shortness of breath. Each question has a 6-point scale (0=“not at all” to 5=“maximal or unable to do because of breathlessness”. The total score ranges from 0 to 120, with higher scores indicating greater dyspnea.

An ANCOVA model with treatment as fixed effect and baseline UCSD-SOBQ score as covariate was applied to observed and imputed data at each visit. Missing results at post-baseline visits were mputed using the predicted values from a random coefficient model. The analysis included all subjects who had baseline and at least one post-baseline UCSD-SOBQ evaluation.

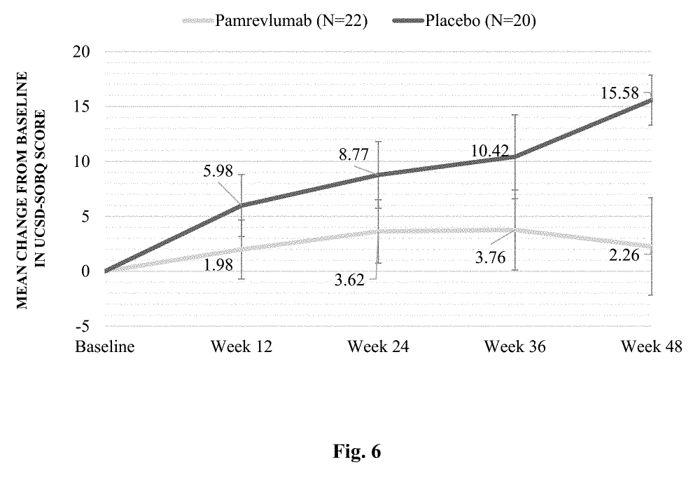

In the pamrevlumab treated group, the mean UCSD-SOBQ score increased slightly to 1.98 at Week 12, that gradually rose to 3.76 at Week 36, before decreasing to 2.26 at Week 48. In contrast, the mean UCSD-SOBQ score for the placebo treated group increases to 5.98 at Week 12 and continued to increase to 15.58 at Week 48. (FIG. 6). The difference of −13.32 points between the two arms was statistically significant in favor of pamrevlumab (p-value=0.0460). The blunted rise and subsequent fall in the mean UCSD-SOBQ score for the pamrevlumab treated group demonstrates the ability of pamrevlumab to arrest the development of worsening dyspnea symptoms in patients with moderate to advanced IPF. To highlight the significance of this finding, no statistical significance was seen for mean UCSD-SOBQ score of pirfenidone treated patients over placebo treated patients at Week 52 in a Phase 3 clinical study (King et al., N Engl J Med 2014; 370:2083-2092).

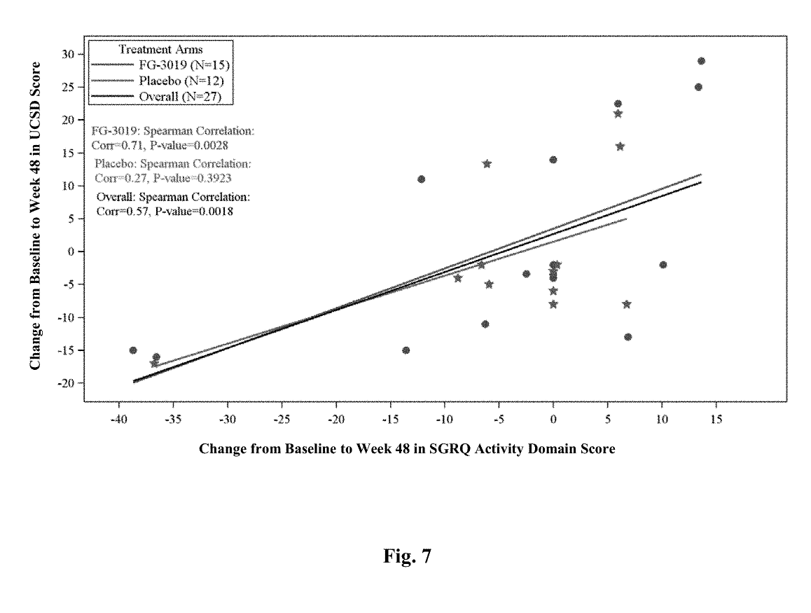

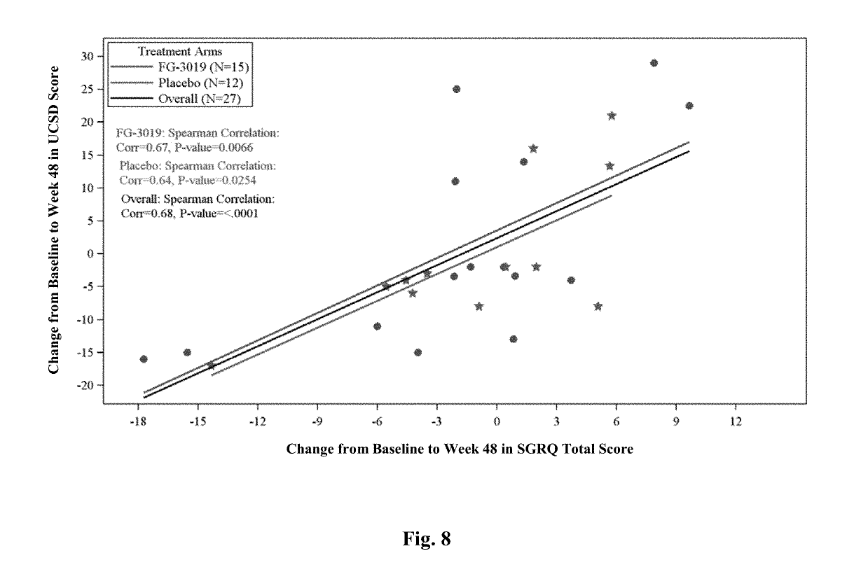

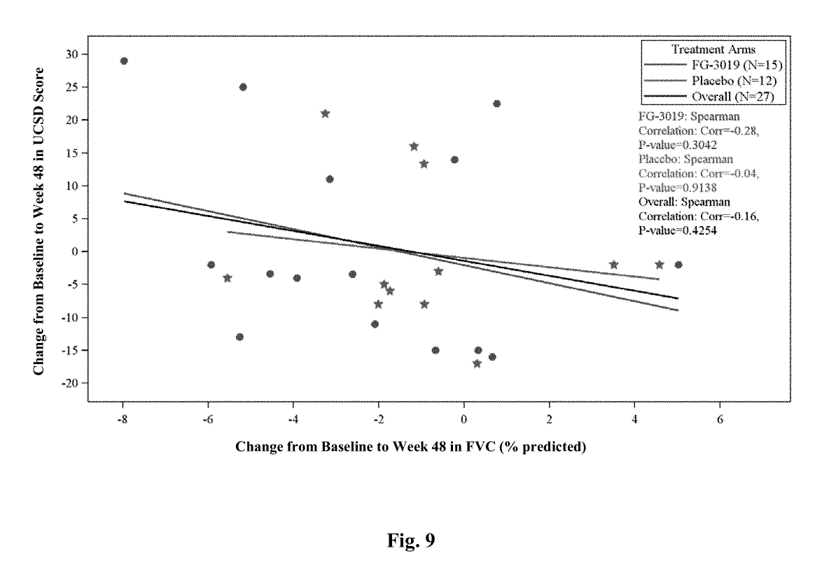

Further, positive correlations were seen between the UCSD-SOBQ scores and the SGRQ activity domain score, the SGRQ total score and FVC %-predicted results (FIGS. 7, 8 and 9).

### Example 4: Occurrence of Common Treatment Emergent Adverse Events

Patients in the Phase two clinical trial described above in Example 2 were monitored for the occurrence of treatment emergent adverse events. The percentage of patients treated with an anti-CTGF antibody (pamrevlumab) that experienced diarrhea was 16.0% compared to 7.5% for placebo treated patients. The percentage of patients treated with pamrevlumab that experienced nausea was 14.0% compared to 13.2% for placebo treated patients. The results demonstrated that the incidence of gastrointestinal toxicities is lower for pamrevlumab treated patients than for that reported for patients in clinical trials of nintedanib (OFEV label), of which 62% experienced diarrhea, compared to 18% for placebo; and 24% experienced nausea compared to 7% for placebo. The incidence of gastrointestinal toxicities is also lower for pamrevlumab treated patients than for that reported for patients in clinical trials of pirfenidone (ESBRIET label) where 26% experienced diarrhea, compared to 20% for placebo; and 36% experienced nausea compared to 16% for placebo.

What is claimed:

1. A method for treating idiopathic pulmonary fibrosis (IPF) in a subject in need thereof, previously treated with pirfenidone and/or nintedanib, the method comprising:
(a) administering to the subject an effective amount of an anti-CTGF antibody, wherein, prior to step (a), pirfenidone and/or nintedanib administration to the patient has been discontinued for at least 2 days, thereby treating IPF.

2. The method of claim 1, wherein the anti-CTGF antibody is pamrevlumab.

3. The method of claim 1, wherein the anti-CTGF antibody has the same amino acid sequence as the antibody produced by the cell line identified by ATCC Accession No. PTA-6006.

4. The method of claim 1, wherein the anti-CTGF antibody binds to CTGF competitively with an antibody produced by the cell line identified by ATCC Accession No. PTA-6006.

5. The method of claim 1, wherein the effective amount of an anti-CTGF antibody is at least about 30 mg/kg.

6. The method of claim 1, wherein the method for treating comprises reducing the pathologic rate of decline of a pulmonary function parameter in the subject.

7. The method of claim 1, wherein the method for treating comprises stabilizing or improving a pulmonary function parameter in the subject.

8. The method of claim 6 or 7, wherein the pulmonary function parameter is selected from the group consisting of vital capacity (VC), residual volume (RV), forced expiratory volume (FEV), forced vital capacity (FVC), forced vital capacity percent predicted (FVCPP), forced expiratory flow (FEF), peak expiratory flow rate (PEFR), inspiratory reserve volume (IRV), functional residual capacity (FRC), inspiratory capacity (IC), total lung capacity (TLC), expiratory reserve volume (ERV), tidal volume (TV), and maximum voluntary ventilation (MVV).

9. The method of claim 1, wherein the method for treating comprises stabilizing or producing at least a 2% reduction, compared to a baseline measurement, in one or more pulmonary radiographic parameters selected from the group consisting of parenchymal fibrosis, ground glass opacities and honeycomb formation.

10. A method of administering an anti-CTGF antibody to a subject in need thereof, comprising administering to the subject a therapeutically effective amount of an anti-CTGF antibody, and avoiding concomitant administration of pirfenidone and/or nintedanib.

11. The method of claim 10, wherein the pirfenidone and/or nintedanib therapy is discontinued at least 2 days prior to starting therapy with the anti-CTGF antibody.

12. A method of treating idiopathic pulmonary fibrosis (IPF) in a subject in need thereof, with an improved gastrointestinal safety profile, comprising administering an effective dose of an anti-connective tissue growth factor (CTGF) antibody, wherein the improved gastrointestinal safety profile of the method is in comparison to current approved IPF therapies (nintedanib and/or pirfenidone).

13. A method for improving the quality of life, stabilizing the rate of decline in the quality of life or reducing the rate of decline in the quality of life of a subject having idiopathic pulmonary fibrosis (IPF) comprising, administering an effective dose of an anti-connective tissue growth factor (CTGF) antibody to the subject, thereby improving the quality of life or reducing the rate of decline in the quality of life of the subject.

14. The method of claim 13, wherein the subject's quality of life is measured by a self-administered questionnaire

15. The method of claim 14, wherein the self-administered questionnaire is the St. Georges Respiratory Questionnaire (SGRQ) or the University of California, San Diego Shortness of Breath Questionnaire (UCSD-SOBQ).

16. The method of any one of claim 10, 12 or 13, wherein the anti-CTGF antibody is pamrevlumab.
